(19) United States
(12) Patent Application Publication
(10) Pub. No.: US 2018/0336381 A1
(43) Pub. Date: Nov. 22, 2018

(54) INTRAVENOUS ACCESS PORT WITH RADIO FREQUENCY BASED LOCATING MEANS

(71) Applicants: Ramin Homanfar, Reno, NV (US); Michael J. Botich, St. George, UT (US)

(72) Inventors: Ramin Homanfar, Reno, NV (US); Michael J. Botich, St. George, UT (US)

(21) Appl. No.: 15/983,081

(22) Filed: May 17, 2018

Related U.S. Application Data
(60) Provisional application No. 62/507,563, filed on May 17, 2017.

(51) Int. Cl. G06K 7/10; G16H 20/17

(52) U.S. Cl. CPC G06K 7/10366; G16H 20/17

(57) ABSTRACT

A radio frequency alignment apparatus and method of use, the radio frequency alignment apparatus having an interrogator with a set of carrier-transmit/data-receive coils, each carrier-transmit/data-receive coils from the set carrier-transmit/data-receive coils configured to have adjustable orientation; the interrogator being configured to emit one interrogation signal within an area of alignment through the set of carrier-transmit/data-receive coils; at least one transponder configured to emit a response signal when energized by the one interrogation signal from the interrogator; the interrogator configured to receive the response signal from the at least one transponder that is processed to generate an indication of a relative orientation of the transponder and the interrogator to each other.

### CROSS-REFERENCES TO RELATED APPLICATIONS

Not Applicable.

### STATEMENT REGARDING FEDERALLY SPONSORED RESEARCH OR DEVELOPMENT

Not Applicable

### REFERENCE TO A “MICROFICHE APPENDIX”

Not Applicable.

### FIELD OF THE INVENTION

The present invention may relate to intravenous ports associated with a radio frequency identification (“RFID”) capability. More particularity to those intravenous ports having associated RFID circuitry that may be used with other RFID instrumentalities to locate the position, orientation, depth and alike factors of a subcutaneous mounted intravenous (IV) port to increase the likelihood of a proper coupling of an external IV needle to the subcutaneous IV port.

### BACKGROUND

The medical procedure known as “infusion therapy” substantially provides liquid or fluid delivery to a patient's blood system (e.g., via a blood vein) through repeated intravenous (IV) connections for those patients undergoing chemotherapy, dialysis or the like. In such circumstances, repeated direct injections in the patient's veins can lead to serious health issues such as vein collapse, systematic infection exposures, necrosis of surrounding tissue (e.g., the chemotherapy pharmaceuticals may cause damage to the patient's tissue when the pharmaceuticals directly contact the tissue.) To offset such issues, the patient may host an artificial injection site such a subcutaneously (e.g., under the patient's skin) located intravenous (IV) port. As substantially shown in FIGS. 1 to 5, a standard IV port 10 may comprise the portal 12, a hollow cup-like housing assembly, the portal 12 further featuring a septum aperture 14 through which projects septum 16, a dome-shaped, re-sealable cap. The hermitically sealed combination of portal 12 and septum 16 forms a hollow interior 18 that is continuously connected to one end of a catheter 20 (e.g., a hollow plastic tube), the other end being connected a subcutaneous IV needle 22 (e.g., a Huber needle having a characteristic bend.) The subcutaneous IV needle 22 penetrates the patient's vein.

The septum 16 may be directed towards the top of the skin 4 so that an external IV needle may pass through the skin 4 and septum 16 to continuously connect to the hollow interior 18. The external IV needle 26 may further feature a Huber or IV needle held by a grip skin pad 30, the pad 30 maybe used for maneuvering the external IV needle 26. This system further allows the contents of an IV bag 34 to be connected by an IV tube 32 to the external IV needle 26. The combination of the IV port 10 and external IV needle 26 allows the liquid contents of the IV bag 34 to be indirectly delivered to the vein 2, sparing the vein 2 the repeated needle puncturing that is otherwise handled by the IV port 10.

Although the subcutaneous IV port generally avoids the issues associated with long term repeated direct vein connection to the IV delivery system, the subcutaneous IV port systems are not without their own set of issues. One possible problem may be the inability to consistently and properly identify the septum's location, orientation, and depth to ensure proper IV needle penetration of the IV port. Lacking proper IV needle contact with the IV port may result in the misplacement or leakage of IV fluid into the tissues surrounding the IV port. In the case of chemotherapy pharmaceutics, this action may also cause necrotic damage to the surrounding tissues and further allowing dislocation of the IV port.

Another possible IV port problem is that the medical treatment being provide through the IV port may cause a physical changes in the patient. A patient's weight change may subsequently causing the amount of skin, adipose tissue or both that are located over the IV port to generally change or may otherwise shift the IV port away from the IV port's original and correct placement under the skin. This physical change could disrupt the IV port's original depth, placement, and orientation to make it harder for the healthcare professionals to properly place the external IV needle into the IV port.

What could be needed is the present invention may be a radio frequency identification (RFID) alignment system and method. Such a system may provide a IV port that is associated with a RFID microchip generally providing the resonance capacitor, a power conditioning circuit, a microcontroller and a coil shunting circuit functionalities (not shown) linked to a carrier-receive/data-transmit coil (e.g., coiled antenna.) A separate RF interrogator (e.g., an RF type transponder) that may be used to detect the subcutaneously located RF IV port could comprise a power source, at least one carrier-transmit/data-receive coil, a resonance capacitor, a carrier transmit drive circuit, an applied data-receive signal detection circuit, various applied data-receive signal filters, various applied data-receive signal amplifier circuits, and various logic devices and/or a microcontroller.

When the RF interrogator power ups its carrier-transmit/data-receive coil(s) could create a first electromagnetic field of flux at a predetermined frequency. When the RF interrogator (and hence first electromagnetic field of flux at a predetermined frequency) the moves proximate to the RF IV port, the RF IV Port's RF microchip becomes passively energized through the first electromagnetic field of flux and self oscillates to create a secondary electromagnetic (EM) field of flux through at a predetermined frequency issued through the RF IV port's carrier-receive/data-transmit coil(s). As the RF interrogator's carrier-transmit/data-receive coil(s) become impressed with RF IV port's return signal (e.g., the secondary electromagnetic field of flux), the RF interrogator's first electromagnetic field of flux is altered. This alteration(s) or backscattering of the first electromagnetic field of flux can be detected by the RF interrogator circuitry to inform the system operator as to closeness of the RF interrogator to the location, orientation and distance to the RF IV port.

### SUMMARY OF ONE EMBODIMENT OF THE INVENTION

Advantages of One or More Embodiments of the Present Invention

The various embodiments of the present invention may, but do not necessarily, achieve one or more of the following advantages:

to provide a IV port with RFID (Radio Frequency Identification) capacity to generate an electromagnetic field of flux that can be utilized to direct an IV needle into the IV port;

the ability to charge an RF (Radio Frequency) microchip with RF energy to create an electromagnetic field of flux proximate to an IV port;

to provide an interrogator to read an electromagnetic field of flux as generated by an IV port to guide the proper intersection of an IV needle into the IV port;

the ability to RF power a RF microchip attached to an IV port to be to create a magnetic induction field with a wire antenna connected to the RF microchip;

to provide an IV port that can be energized by RF energy to create an electromagnetic field whose flux may be used by an RF interrogator to correctly guide place an external IV needle into proper contact with a subcutaneously mounted IV port; and

the ability to determine the location, orientation, position and relative distance of a subcutaneous placed IV port using RFID based means to guide an external IV needle into subcutaneous placed IV port.

These and other advantages may be realized by reference to the remaining portions of the specification, claims, and abstract.

### BRIEF DESCRIPTION OF THE DRAWINGS

FIG. 1 is substantially a perspective cutaway view of an intravenous port system.

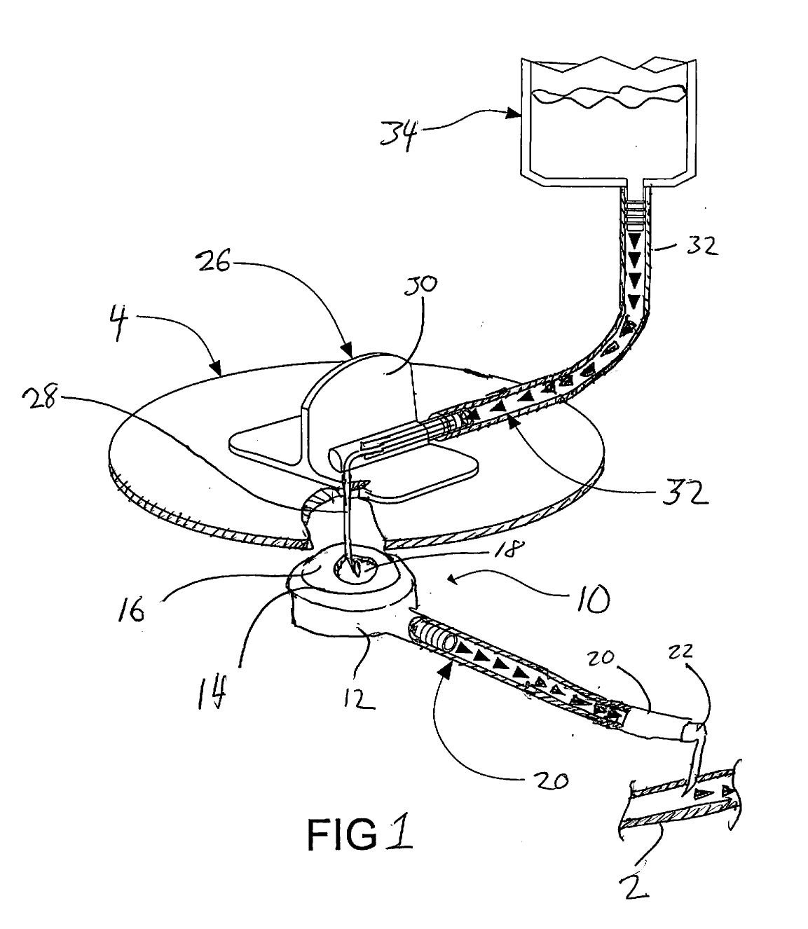

FIG. 2 is substantially a perspective cutaway view of an intravenous port and intravenous needle.

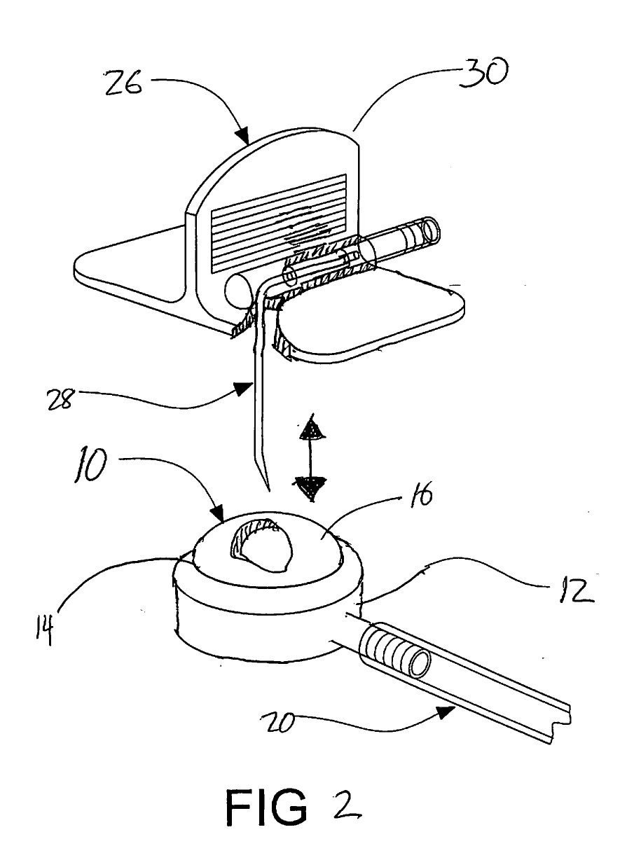

FIG. 3 is substantially a perspective cutaway view of an external intravenous needle.

FIG. 4 is substantially a top view of an intravenous port.

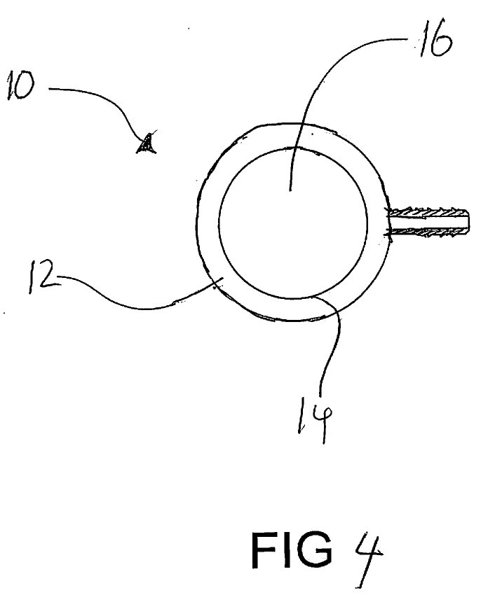

FIG. 5 is substantially a side elevation cutaway view of an intravenous port.

FIG. 6 is substantially a side elevation cutaway view of RF intravenous port of one embodiment of the invention.

FIG. 7 is substantially a perspective cutaway view of an external intravenous needle engaging RF intravenous port of one embodiment of the invention.

FIG. 8 is substantially a perspective view of the RF microchip and coil.

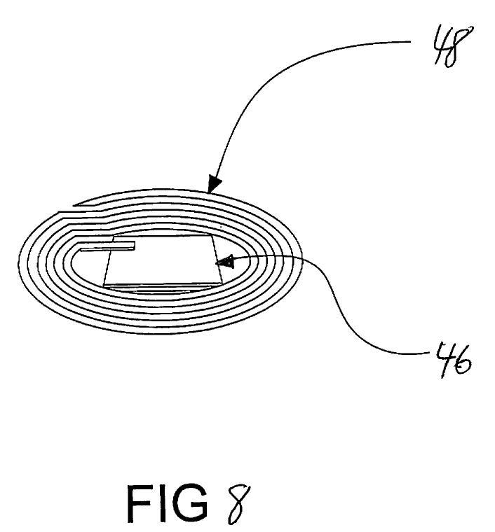

FIG. 9 is substantially a perspective view of the RF microchip and plug combination.

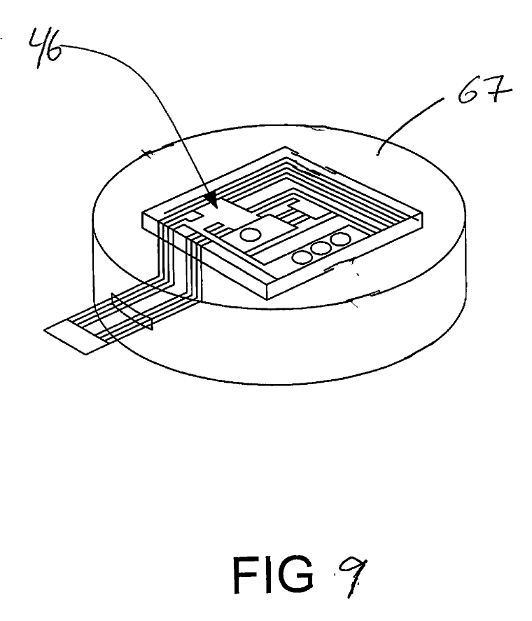

FIG. 10 is substantially an exploded perspective view of another embodiment of RF intravenous port.

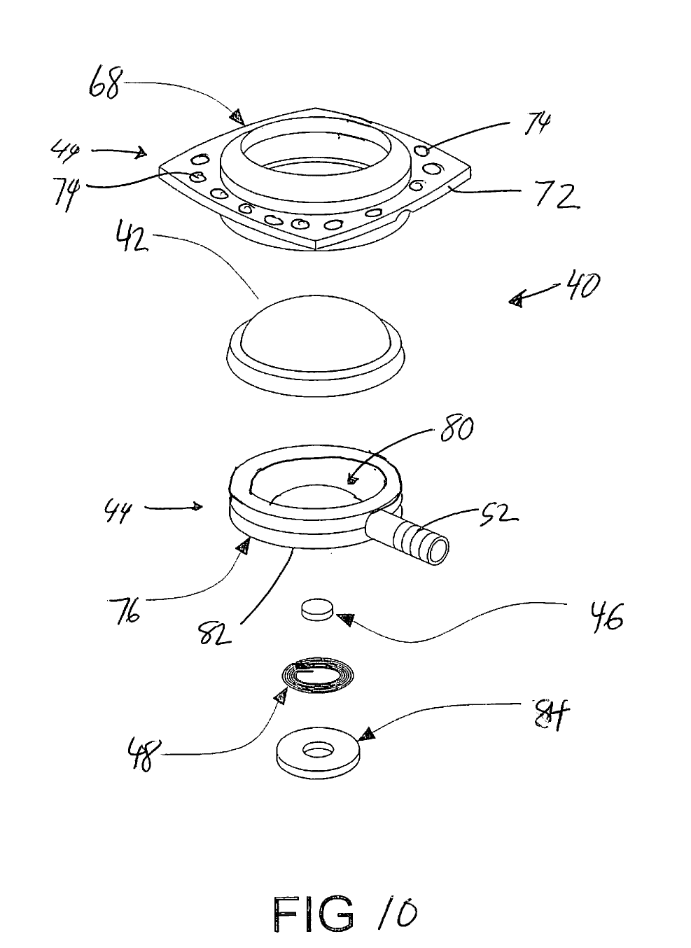

FIG. 11 is substantially a cutaway perspective view of the external intravenous needle, the RF intravenous port and the RF interrogator.

FIG. 12 is substantially a cutaway perspective view of the external intravenous needle, the RF intravenous port and the RF interrogator.

FIG. 13 is substantially an end elevation cutaway view of the RF interrogator of the external intravenous needle, the RF intravenous port and the RF interrogator showing first and second electromagnetic fields of flux.

### DESCRIPTION OF CERTAIN EMBODIMENTS OF THE PRESENT INVENTION

In the following detailed description of the preferred embodiments, reference is made to the accompanying drawings, which form a part of this application. The drawings show, by way of illustration, specific embodiments in which the invention may be practiced. It is to be understood that other embodiments may be utilized and structural changes may be made without departing from the scope of the present invention.

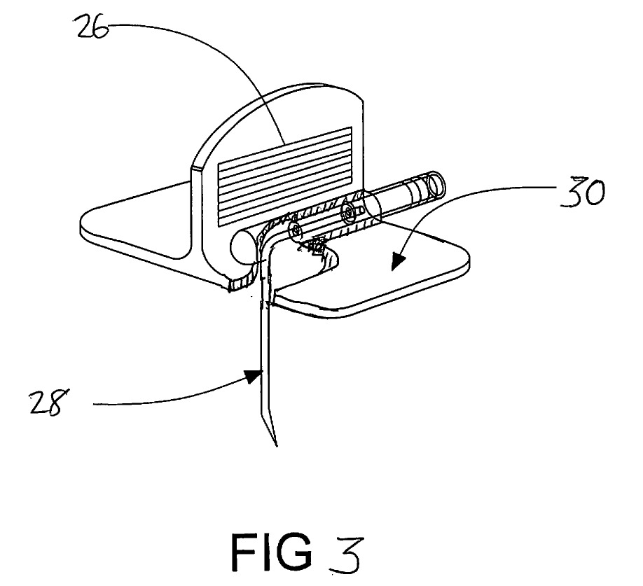

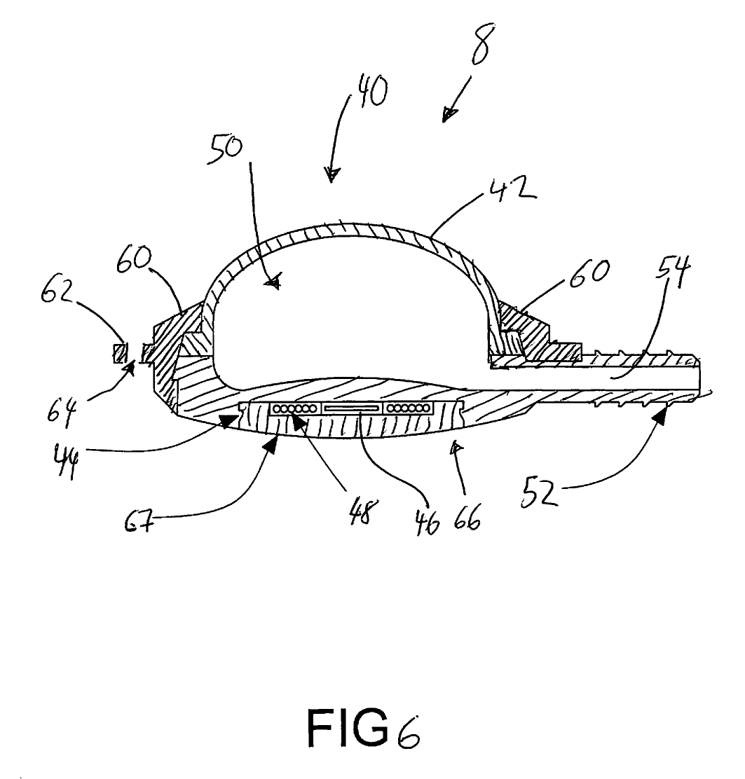

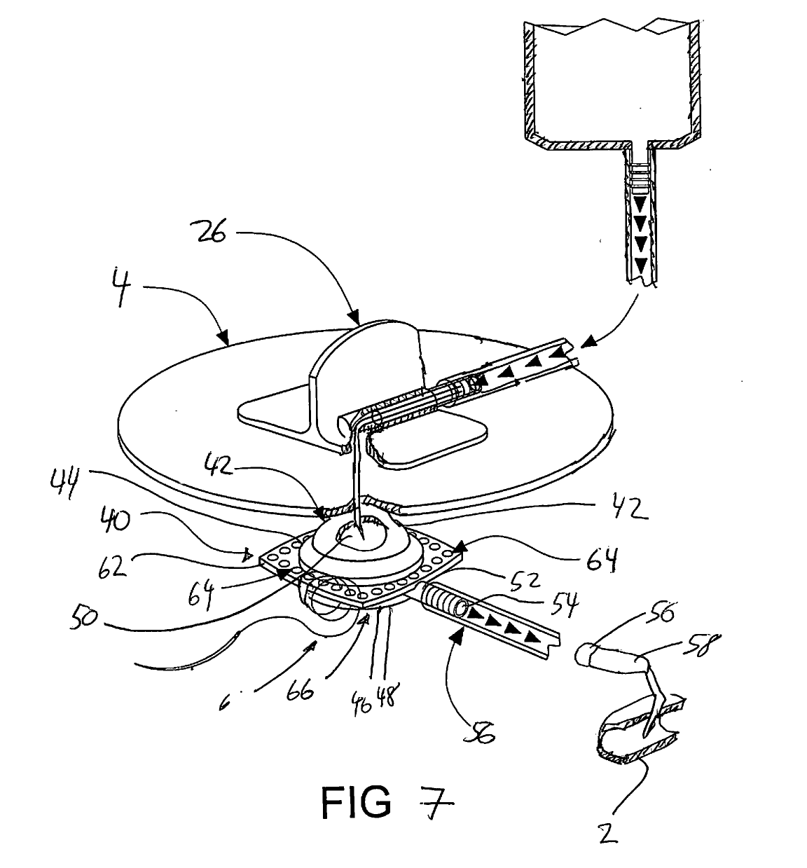

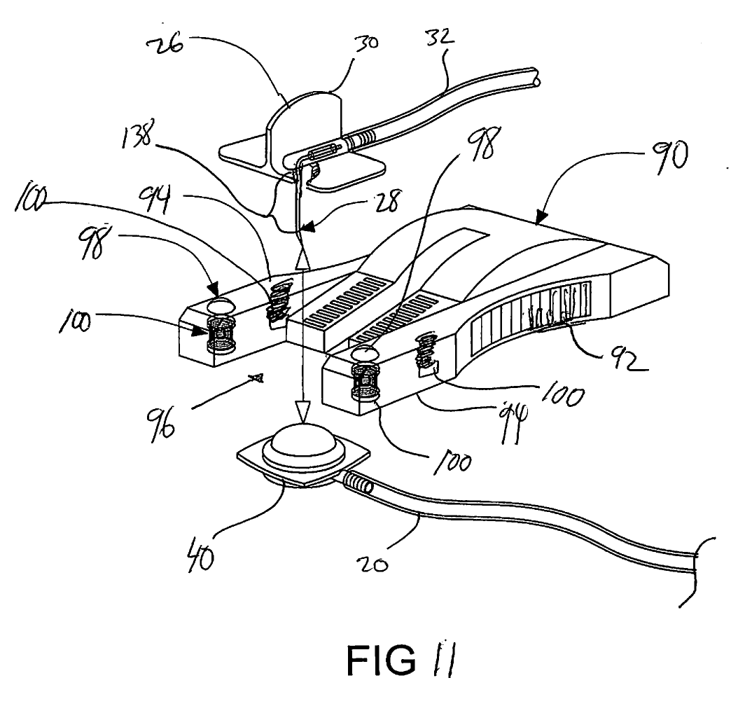

The present invention 8 may be a non-contact RF transducer alignment function and system utilizing Radio Frequency Identification (“RFID”) technology. The system could comprise an RF (Radio Frequency) IV (IntraVenous) port 40 (as substantially shown in FIGS. 6-7) and a RF interrogator 90 (as substantially shown in FIGS. 11-13). When the energized RF interrogator 90 creates a first electromagnetic field of flux at a predetermined frequency that is received by the RF IV Port 40, the RF IV port 40 passively energizes to subsequently generate and issue a second electromagnetic field of flux at a predetermined frequency. When second electromagnetic field of flux at a predetermined frequency reaches the RF interrogator 90, second electromagnetic field of flux alters (backscatters) the first electromagnetic field of flux. The RF interrogator 90 is able to measure the backscatter to determine the location of the RF IV port 40 relative to the RF interrogator 90 and to inform the system operator (not shown) how to move and locate the RF interrogator 90 over the RF IV port 40. The RF interrogator 90 once so located over the RF IV port 40 can then generally direct an IV or Huber needle 26 (as substantially shown in FIG. 3) into continuous communication with hollow interior 50 of the subcutaneous RF IV port 40.

As substantially shown in FIGS. 6 and 7, the RF IV port 40 could comprise a septum 42, a portal 44 and a RF microchip 46 connected to a carrier-receive/data-transmit coil 48 (e.g., a transponder.) The septum 42, a re-sealable, flexible hemispherical or dome-shaped membrane, may be hermetically fixed to the portal 44, a cup-shaped housing assembly, to substantially form a hollow receiving interior 50. The portal 44 could externally provide an open-ended barbed projection 52 with a hollow channel 54 that is continuously connected to the hollow receiving interior 50. The barbed projection could mount a catheter 56 (as substantially shown in FIG. 7). The catheter 56 could be attached at one open end to the barbed projection 52 while subcutaneous a Huber or IV needle 58 could be attached to the other open end of the flexible catheter 56 to removably and continuously attach the catheter 56 to a patient's vein 2. An edge of the septum 42 could be held in a compressed sealed connection to the portal 44 by virtue of a snap fit of a retention ring 60 as applied to the portal 44. Protruding around the perimeter of the portal 44 could be a flange 62 with suture apertures 64 used to suture the IV RF port 40 in a secure position within the patient's tissue.

The underside of the portal 44 could form a recess 66 to accommodate the placement of the RF microchip 46 connected to a carrier-receive/data-transmit coil 48. A snap-fit disc cover 66 could lock into the recess 67 to otherwise generally enclose and hold the suitable RF microchip 46 and its associated carrier-receive/data-transmit coil 48 in place in the recess 67. The RF microchip 46 could comprise semiconductor functionalities (not shown) of a resonance capacitor, a power conditioning circuit, a microcontroller, and a coil shunting circuit.

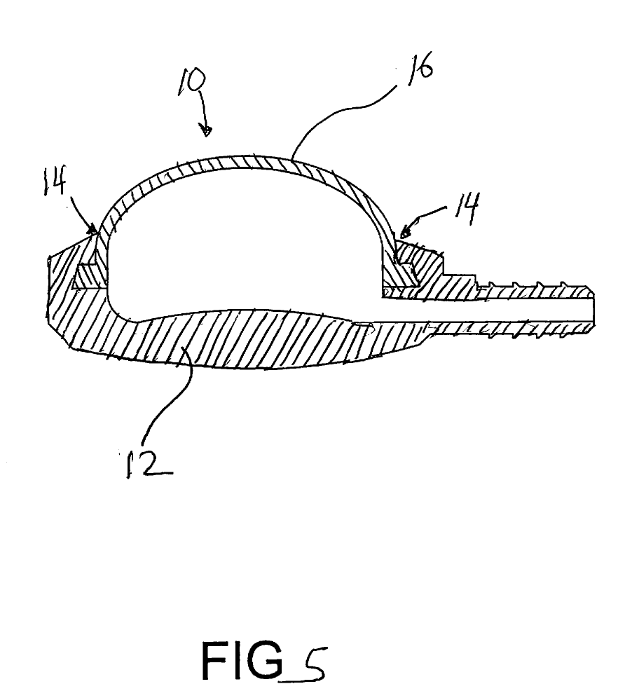

As substantially shown in FIG. 5, one possible embodiment of the RF IV port 40 could have a portal 44 as an assembly generally comprising a top ring 68 with an integrated suture flange 72 forming suture apertures 74 and a cup-shaped portal base 70 further forming a barbed protrusion 78. The septum 42 generally sits on atop the portal base 70 while the top ring 70 descends over the edge of the septum 42 to clip-fit on to the portal base 70 to hermitically seal the septum 42 to the portal base 70 and thereby substantially forming the hollow interior 80. The RF microchip 46 and associated carrier-receive/data-transmit coil 48 could be nested in a recess 82 in the bottom of the portal base 70 and be covered by a disc cover 84.

Many if not all of the materials used in the RF IV port 40 could be bio-compatible with the patient's tissue. For example, the septum 42 could be made out of a silicone material while rigid components of the RF IV port 40 could be formed from polycarbonate or other suitable polymers used in implantation.

As substantially shown in FIG. 11, the RF interrogator 90 could be configured with generally flat U-shape with a grip 92 having a recessed sides, the grip 92 connecting two arms 94 held in spaced-apart parallel orientation forming an RF IV portal receiving dock 96. The RF IV portal receiving dock 96 could receive and position the external IV needle 26 once the RF interrogator 90 has located the subcutaneous RF IV port 40 between the both arms 94. The RF interrogator 90 may further comprise one or more indicator lights 96 (e.g., LEDs) generally connected to the remainder of interrogator circuitry. The indicator lights 96 may transmit information to the system operator (not shown) as to the orientation, position and depth of the subcutaneous location of the subcutaneous RF IV port 40 so the operator may move the RF interrogator 90 directly over the RF IV port 40. Once so located, the exterior IV needle 26 could be removably received within the RF IV portal receiving dock 96 to properly connect to further connected to and pierce the septum 14 as the exterior IV needle's grip pad 30 is brought into contact with the patient's skin 4.

In one possible embodiment, the remainder RF interrogator circuitry (not shown) could further include a power source (e.g., battery [DC], wall socket [AC] or other) for powering RF transponder device, at least one carrier-transmit/data-receive coil, a resonance capacitor, a carrier transmit drive circuit, an applied data-receive signal detection circuit, various applied data-receive signal filters, various applied data-receive signal amplifier circuits, and various logic devices and/or a microcontroller wherein the RF IV port 40 and RF interrogator 90 act together, create a non-contact, RF transducer alignment function and system.

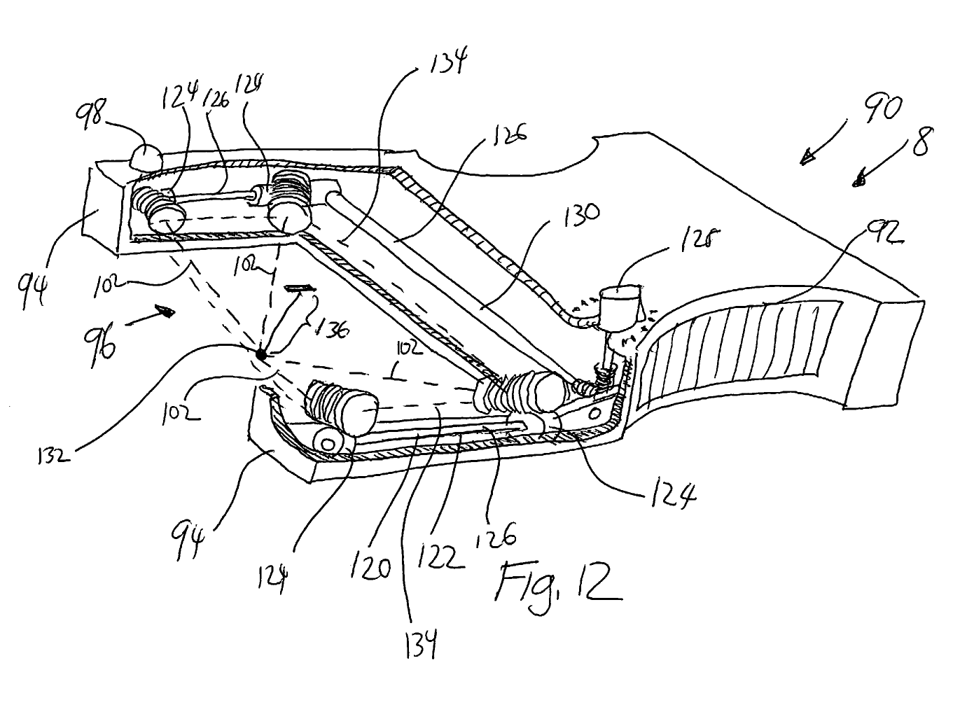

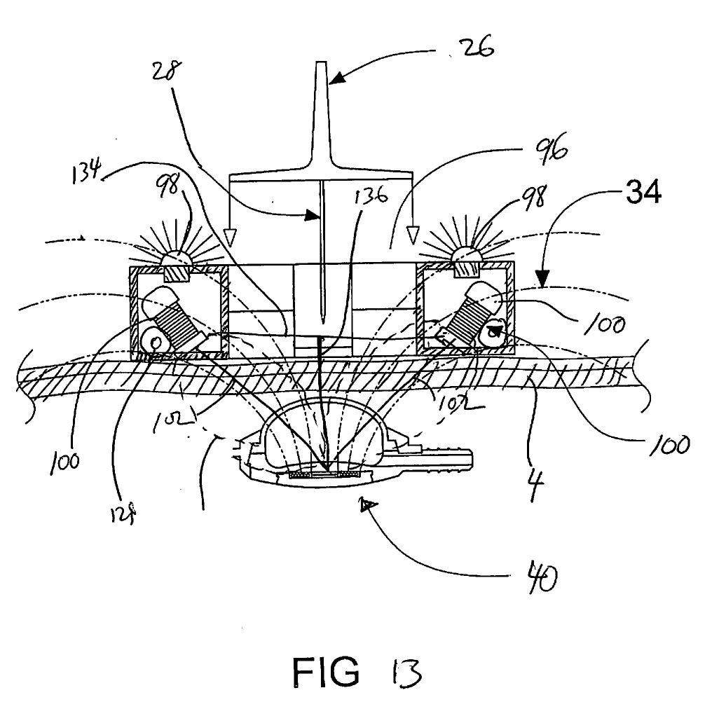

In one possible embodiment as substantially shown in FIGS. 12 and 13, the RF interrogator 90 could have four carrier-transmit/data-receive coils 100, two in spaced-apart relationship in each arm 94 (one coil in the tip and another coil in the base of the arm 94) so the respective carrier-transmit/data-receive coils 48 are distanced from each other to substantially form the corners of a square. The carrier-transmit/data-receive coils 94 may be further connected together by a mutual angling apparatus 120 so that the movement of carrier-transmit/data-receive coils 100 within the arms 94 may synchronized so that center line 102 orientation of each coil carrier-transmit/data-receive coils 100 can be angled downwards to intersect one another to geometrically form a tip of a pyramid that is aimed below the RF interrogator 90. As RF interrogator 90 issues the first electromagnetic field flux 136 from its carrier-receive/data-transmit coils 100, the intercept angling of RF interrogator's carrier-transmit/data-receive coils centerlines 102 may allow the RF interrogator 90 to correctly interpret the distance between the RF interrogator 90 and the RF IV portal 40 as that distance relates to the distance from the intercept point 132 to the interrogator plane 134 set by the location and orientation of the carrier-transmit/data-receive coils 100 within the arms 94. As the carrier-transmit/data-receive coils 100 are further mutually angled downward, increasing the length of the distance 136 between the intercept point and the plane, this action may also increase the measured distance that the RF interrogator 90 will inform the system operator as to where RF IV portal 40 is located. In this manner, the RF interrogator 90 can be adjusted to find the depth of the RF IV portal 40 that corresponds to the operative length 138 (as substantially shown in FIG. 11) of the selected external IV needle 26.

One possible embodiment of the mutual angling apparatus 120 could be an axle-gear box combination 122. In this manner, each carrier-transmit/data-receive coil 100 is connected to and moved by a respective gear box 124. These gearboxes 124 could be connected together by a set of axles 126, with one of the axles being further geared to interact with a geared knob 128 as movably supported by the RF interrogator's case. Rotation movement applied to the geared knob 128 could be imparted to geared axle 130 to be passed along to gear boxes 124 and remaining axles 126 enabling them to change the positioning of the carrier-transmit/data-receive coils' orientations in synchronized way. In this manner, the centerlines 102 of the carrier-receive/data-transmit coil 100 can angle downward to the intersect point 132 to generally form an intersecting tip of a pyramid extending from four pyramid corners are presented by the location of respective carrier-transmit/data-receive coils 100 of the RF interrogator 90. Case markings that are proximate to the geared knob 128, can be used with the geared knob rotation to indicate when the proper intercept point-to-plane distance length 136 is set relative to the selected external IV needle's operative length 94. In another version not shown, each carrier-transmit/data-receive coil may be connected to and moved by a respective servo whose activation and movement are controlled by the interrogator's circuitry.

In one possible detection method embodiment, the geared knob is adjusted to set the distance sensitivity upon which the RF interrogator that will activate the LEDs in proximity to the subcutaneous RF IV port. RF interrogator's carrier transmit/data receive coils produces a first electromagnetic field of flux at a predetermined frequency. When the first electromagnetic field of flux moves to close proximity the RF IV port, the RF IV portal energizes or “powers up”. As the RF IV port's RFID microchip is passively charged-up in this manner, the RFID microchip may cause the associated the carrier-receive/data-transmit coil to emit a second electromagnetic field of flux. The second electromagnetic field of flux at a predetermined frequency interacts with and alters first electromagnetic field of flux at a predetermined frequency to create a backscattering. The RF interrogator circuitry may read the backscattering to determine the position, orientation and distance of the subcutaneous RF IV port relative to the current position of the RF interrogator. The RF interrogator circuitry may also substantially relay these parameters to the system operator by activating the lights in a manner that the operator can understand where the RF interrogator's position in relation to a subcutaneously mounted RF IV port. Through the light transmission (e.g., moving from a blinking to solid on illumination), the RF interrogator can guide the operator's movement of the RF interrogator to locate the RF interrogator directly over the subcutaneous RF IV port so the operator can place the external IV needle into the RF IV portal receiving dock to locate the tip of the IV needle to penetrate into the septum and connect with the hollow receiving interior.

### CONCLUSION

Although the description above contains many specifications, these should not be construed as limiting the scope of the invention but as merely providing illustrations of some of the presently preferred embodiments of this invention. Thus, the scope of the invention should be determined by the appended claims and their legal equivalents rather than by the examples given.

What is claimed is:

1. A radio frequency alignment apparatus comprising:
(A) an interrogator having a set of carrier-transmit/data-receive coils, each carrier-transmit/data-receive coil from the set carrier-transmit/data-receive coils is configured to have adjustable orientation to one another; the interrogator being further configured to emit one interrogation signal within an area of alignment through the set of carrier-transmit/data-receive coils;
(B) at least one transponder configured to emit a radio frequency signal when receiving the one interrogation signal from the interrogator;
(C) the interrogator configured to receive the radio frequency signal from the at least one transponder and process the received radio frequency signal to generate an indication of a relative orientation of the transponder and the interrogator to each other.

2. The apparatus of claim 1 wherein the carrier-transmit/data-receive coils are further interlocked to simultaneously adjusting their respective orientations to one another.

3. The apparatus of claim 2 wherein the interlock capability is mechanically accomplished.

4. The apparatus of claim 2 wherein the interlock capability is electronically accomplished.

5. The apparatus of claim 1 wherein a length of a distance of detection between the interrogator and the transponder is altered by a change in the orientations of the carrier-transmit/data-receive coils.

6. The apparatus of claim 5 wherein the length of a distance of detection corresponds to an operative length of an external intravenous needle.

7. The apparatus of claim 5 wherein the length of a distance of detection corresponds to a second length of distance between an intersection of centerlines of the carrier-transmit/data-receive coils to a plane of the interrogator.

8. The apparatus of claim 1 wherein any change in a length of a distance from an intersection of centerlines of the carrier-transmit/data-receive coils to a respective carrier-transmit/data-receive coil during the orientation adjustments for the set of carrier-transmit/data-receive coils is the same for each carrier-transmit/data-receive coil.

9. The apparatus of claim 1 wherein the transponder is attached to the intravenous port.

10. The apparatus of claim 1 wherein the interrogator is configured to have a pair of arms mounting the set of carrier-transmit/data-receive coils, the pair of arms are further configured to form a dock that removably receives an intravenous needle.

11. The apparatus of claim 10 wherein each arm has one carrier-transmit/data-receive coil in the tip of the arm and another carrier-transmit/data-receive coil in the base of the arm.

12. A method of aligning a plurality of devices comprising:
(A) adjusting orientations of interrogator carrier-transmit/data-receive coils relative to each other;
(B) emitting from an interrogator that is configured to associate with a first device an electromagnetic field of flux;
(C) emitting a radio frequency signal from a transponder associated with a second device when the transponder is in proximity to the electromagnetic field of flux from the interrogator;
(D) detecting the radio frequency signal from the transponder when the interrogator is to in a condition of alignment with the second device; and
(E) indicating when the radio frequency signal from the transponder is detected.

13. The method of claim 12 further selecting the adjustments to be made to the orientations of interrogator carrier-transmit/data-receive coils based on the operative length of a selected intravenous needle.

14. The method of claim 12 wherein the adjusting orientations of interrogator carrier-transmit/data-receive coils further comprises a step of intersecting the centerlines of the interrogator carrier-transmit/data-receive coils to a point outside of a plane of the interrogator.

15. The method of claim 14 further comprises a step of setting lengths of the distances from the centerline intersection point to respective interrogator carrier-transmit/data-receive coils to be the same.

16. The method of claim 12 wherein the step of intersecting the centerlines of the interrogator carrier-transmit/data-receive coils further comprises a step of defining a distance between the intersection point and a plane that contains interrogator carrier-transmit/data-receive coils, a length of the distance is related to an operative length of a selected intravenous needle.

17. The method of claim 12 further comprises a step of locating the interrogator proximate to the second device.

18. The method of claim 17 further comprises a step of directly connecting the first device to the second device by placing the first device in direct contact with the interrogator.

19. The method of claim 18 further comprises a step penetrating the second device with the first device.

20. The method of claim 10 further comprises a step of subcutaneously mounting second device and transponder together.
